(19) United States
(12) Patent Application Publication
(10) Pub. No.: US 2011/0104282 A1
(43) Pub. Date: May 5, 2011

(54) New Therapy of Treatment of the Irritable Bowel Syndrome

(75) Inventors: Greger Lindberg, Stockholm (SE); Aldona Dlugosz, Alta (SE)

(73) Assignee: Karolinska Institutet Innovations AB

(21) Appl. No.: 12/989,447

(22) PCT Filed: Apr. 27, 2009
(86) PCT No.: PCT/SE09/50440

§ 371 Date: Jan. 14, 2011

Related U.S. Application Data
(60) Provisional application No. 61/047,843, filed on Apr. 25, 2008.

(51) Int. Cl. A61K 9/00; A61K 31/56; A61K 31/405; A61K 31/573; A61K 31/58; A61K 31/7052; A61K 31/7048; A61K 31/5383; A61K 9/16; G01N 33/68; A61P 1/00

(52) U.S. Cl. 424/486; 514/171; 514/419; 514/29; 514/229.5; 424/490; 424/484; 424/400; 435/7.32; 436/501

(57) ABSTRACT

The invention provides for new methods for treatment and diagnosis of irritable bowel syndrome (IBS). In particular, there is disclosed the use of a Chlamydia activating agent and one or several antibiotics selected from macroildes and benzoxazinorifamycins in the preparation of combination agent for simultaneous, concomitant or sequential administration for the treatment of IBS.

### FIELD OF INVENTION

The present invention relates to a new therapy of treatment of the irritable bowel syndrome.

### BACKGROUND OF INVENTION

The irritable bowel syndrome (IBS) is a common disorder that may affect as many as 9%-15% of the population in Western countries.¹⁻³. Sometimes irritable bowel syndrome is referred to as spastic colon, mucous colitis, spastic colitis, nervous stomach, or irritable colon. Generally, IBS is classified as a “functional” disorder. IBS is characterized by abdominal pain and disturbed bowel function in absence of a detectable organic disease that would explain symptoms.⁴ Causality in IBS has remained enigmatic. Ever since the first attempts to systematically describe IBS^{5,6}, emphasis has been put on the frequent co-existence of bowel symptoms and psychosocial factors. However, a true cause-effect relationship between psychosocial factors and IBS was never established.^{7,8}

The presence of disturbed gut function indicates that the control systems of the gut are involved in the pathogenesis of IBS. The control systems are the enteric nervous system, the network of interstitial cells of Cajal, and the enteroendocrine cells. Due to its location, the enteric nervous system is difficult to study. Previous studies have revealed lymphocytic infiltration and neuron damage in myenteric ganglia when full-thickness biopsies from the jejunum in 10 patients with severe IBS were investigated⁹. The presence of lymphocytes suggests that immune activation may be involved in the pathogenesis of IBS. The cause of immune activation remains uncertain. Recent observations have also indicated that so called post-infectious IBS can arise after almost any type of acute gastroenteritis. However, the actual agent causing gastroenteritis was not a predictor of risk for IBS.¹⁰ Consequently, a host factor might influence the development of IBS.

One previous observation is that IBS occurs in 35%-50% of females with the chronic pelvic pain syndrome.¹¹⁻¹³ In addition, results from animal experiments indicate that the small bowel is a possible reservoir for Chlamydia.¹⁴ However, a previous attempt to link C. trachomatis to IBS using serum IgG antibodies failed.¹⁵ It appears that IgG antibody patterns are insufficient to rule out persistence of C. trachomatis as well as persistence of any other Chlamydia as driving factors for immune activation^{16,17}.

Today, diagnosis of IBS is carried out by classifying symptoms along standard criteria (Rome III Criteria). Usually diagnosis is made by making the patient answer a number of questions relating to bowel function. The exclusion of other kinds of bowel disease is also a major method of diagnosis. Thus, there is a lack of clear-cut and objective methods for diagnosis of IBS.

The present inventors have by investigation found that persistence of Chlamydia in the gut has instrumental role of the pathology in IBS and there is an association between persistent Chlamydia infection in the small bowel and IBS. It has also been found that Chlamydia trachomatis in its persistent form is present in enteroendocrine cells and in this stage would be capable of triggering the disorders associated with IBS. It has previously been suggested by M Singla in Medical Hypothesis, 2007, 68, 278-280, that chronic infections from Chlamydia trachomatis potentially may be treated with a combination of tryptophan and an antibiotic: A supplementation of tryptophan before therapy with antibiotics is suggested to make the organism more susceptible and decrease the development of resistant forms. However, this disclosure does not give any guidance to an effective therapy against IBS.

It is therefore an object of the present invention to outline an effective therapy against IBS based on the surprising finding that Chlamydia trachomatis in its persistant form is associated with such disorders.

Another object of the present invention is to provide a method for diagnosis of IBS based on the said findings of Chlamydia trachomatis in such disorders.

### DESCRIPTION OF INVENTION

The present invention relates to the use of a Chlamydia activating agent and one or several antibiotics selected from macrolides and benzoxazinorifamycins in the preparation of combination agent for simultaneous, concomitant or sequential administration for the treatment of the irritable bowel syndrome (IBS) (colon irritabile). The present invention relates to a method of treating IBS by administering a combination agent comprising a Chlamydia activating agent and one or several antibiotics selected from macrolides and benzoxazinorifamycins. A combination agent in the meaning of the present invention is any manner of combining administration of a Chlamydia activating agent and administration of an antibiotic with the purpose of treating IBS. The combination agent can be a unit preparation or a preparation comprising the constituents of a Chlamydia activating agent and an antibiotic separately for simultaneous, concomitant or sequential administration according to a predetermined regimen. The combination agent can further be provided in the form of a kit with a regimen to administer Chlamydia activating agent and antibiotic in according to a regimen, such as a pre-treatment period with said activating agent and a following antibiotic treatment period, where both periods have preset duration and dosing.

A Chlamydia activating agent in the meaning of the present invention has a capacity to affect Chlamydia-type organisms to transfer from its persistent form within host cells to its metabolically active form associated with an inflammatory response and the complications associated with a Chlamydia infection. It is usually considered that Chlamydia is resistant to antibiotic treatment during its persistent stage.

In one embodiment the Chlamydia activating agent is tryptophan. In another embodiment the Chlamydia activating agent is a steroid, preferably selected among cortisone, prednisolone, budesonide and similar steroid agents conventionally used as immunosuppressants. It is also conceivable to combine administration of tryptophane and one or more steroid as a Chlamydia activating agent in a Chlamydia activating step. In other terms, the Chlamydia activating agent can be mixture of tryptophane and at least one steroid or different dose forms of tryptophane and at least one steroid for separate administration in a predetermined sequence.

Macrolides are antibiotics, often produced by various strains of Streptomyces, and widely used in a clinical aspect for infectious diseases caused by gram-positive bacteria, some kinds of gram-negative bacteria, mycoplasma and Chlamydia due to their strong anti-bacterial activity. Macrolides, such as erythromycin and roxithromycin, are antibiotics formed by binding of a lactone ring (often a 14-membered ring) to side chains such as a methyl chain or dimethylamino sugar or a neutral sugar. Their mechanism of action is to affect the 50S subunit of the ribosome of bacteria and to inhibit the reaction of transpeptidase activity so as to suppress protein synthesis. Examples of useful macrolides include, without any limitation, erythromycin, oleandomycin, troleandomycin, roxithromycin, clarithromycin, davercin, azithromycin, flurithromycin, dirithromycin, josamycin, spiromycin, midecamycin, loratadine, desloratadine, leucomycin, miocamycin, rokitamycin, andazithromycin, and swinolide A.

Benzoxazinorifamycins, as exemplified by rifalazil, are rifamycins that contain a distinct planar benzoxazine ring providing them with specific physical and chemical characteristics. Rifalazil has strong antibacterial activity, high intracellular levels and high tissue penetration, and is thereby highly useful for treating diseases caused by Chlamydia. These agents inhibit bacterial DNA-dependent RNA polymerase. The benzoxazine ring provides benzoxazinorifamycins with unique physical and chemical characteristics which favor the use of rifalazil and derivatives in treating diseases caused by the obligate intracellular pathogens of the genus Chlamydia. Useful benzoxazinorifamycins in the present context are further described by D M Rothstein et al in Arch Pharm (Weinheim). 2007 October; 340(10):517-29.

Useful antibiotics among macrolides and benzoxazinorifamycins can, for example, be selected among drugs presently prescribed or contemplated for urogenital diseases caused by Chlamydia trachomatis.

In the present context, a combination agent may comprise an orally administrable dose form where the two agents are combined to a unitary dose of each agent in a solution, tablet, capsule or the similar, either by admixture or in different compartments. A combination agent may alternatively be physically different dose forms for oral administration in a kit or the similar where different units of the two agents are provided together with an administration regimen for the sequence or timing for administration in a concomitant or sequential manner.

The present invention depends on a pre-treatment period with the Chlamydia activating agent and that the combination agent is adapted for this purpose. The pre-treatment period extends to 3 to 7 days, preferably 3 days, when the activating agent is administered on daily basis. For tryptophan a suitable pre-treatment oral dosing is 50 to 500 mg four times a day and preferably 250 mg four times a day, most preferably administered for at least 3 days. For steroids, a suitable oral dosing aligns with doses conventionally recommended for immunosuppression. For prednsiolone suitable doses are 15-30 mg/day and for budesonid 3-9 mg/day.

Following the pre-treatment period, the present invention depends on a period of treatment with antibiotics of the combination agent, either adjunct with the activating agent administered in the same manner as during the pre-treatment period, or in the absence of the activating agent. Suitable doses of macrolide or benzoxazinorifamycins suitable substantially align with what is conventionally prescribed for treatment of Chlamydia infections. Suitably arithromycin is administered with a total weekly dose of 1000 mg for 8-12 weeks, erythromycin is administered in 500 mg twice daily for 4 weeks and rifalazil is administered with a weekly dose of 25 mg for 8-12 weeks. In one aspect of the invention, 250 mg/day of tryptophane is adjunctively administered or coadministered with the macrolide or benzoxazinorifamycins. Preferably the antibiotics are administered in the mentioned doses. During the treatment period an antibiotic selected among macrolides or benzoxazinorifamycins is administered in suitable or conventional doses for Chlamydia treatment. Alternatively, when regarded clinically relevant or necessary, two antibiotics can be administered during the treatment period. Preferably, in this case a macrolide and a benzoxazinorifamycin are co-administered and most preferably erythromycin and rifalazil, suitable in the described dose regimens.

A suitable regimen would comprise a pre-treatment period with 250 mg tryptophane four times a day during 3 days and subsequently an adjunct therapy with tryptophan 250 mg/day and rifalazil 25 mg/week during 8-12 weeks.

Since the entire intestinal tract, i.e. the duodenum, jejunum, ileum and colon, is observed to host Chlamydia type organisms it is advantageous to deliver the Chlamydia activating agent to the entire intestinal tract i.e. to expose the cells hosting the Chlamydia organisms. The transit time through the GI-tract (gastrointestinal tract) can be up to 30 hours. An average transit time is said to be about 22 hours. For this reason, it is a special embodiment of the invention to employ a delivery system, i.e. a capsule or tablet that can release the Chlamydia activating agent during a period as close as possible to 22 hours. By this kind of delivery system, i.e. an extended release oral formulation the following benefits would be achieved compared to conventional tablets and capsules.

The rate of drug release for a Chlamydia activating agent, e.g. tryptophan, can be modified by the following suitable technologies: (a) modifying drug dissolution by controlling access of biological fluids to the drug through the use of barrier coatings, and (b) controlling drug diffusion rates from dosage forms.

A first suitable extended release system for a Chlamydia activating agent is directed to barrier coatings based on coated beads, granules and/or microspheres. In these systems the Chlamydia activating agent is distributed onto beads, granules or other particulate systems. Using conventional pan coating or air suspension coating, a solution of the drug substance is placed on small inert seeds or beads made of sugar or starch or on microcrystalline cellulose spheres. When the dose is large some of the granules may remain uncoated to provide immediate drug release. Other granules (about two-thirds to three-fourths) receives varying coats of lipid material like beeswax, carnuba wax, glyceryl monostearate, cetyl alcohol or a cellulosic material like ethylcellulose. Various commercial aqueous coating systems include ethyl cellulose and plasticizer as the coating material (e.g. Aquacoat (FMC Corporation) and Surelease (Colorcon). The variation in the thickness of the coats and in the type of coating material used affects the rate at which body fluids penetrate the coating to dissolve the drug. Granules of different coating thickness are blended to achieve a mix having the desired drug-release characteristics. When properly blended, the granules can be placed in capsules or compressed to tablets.

A second suitable extended release system for a Chlamydia activating agent is directed to embedding drug in a slowly eroding or hydrophilic matrix system. The Chlamydia activating agent is combined and made into granules with an excipient material that slowly erodes in body fluids, progressively releasing the drug for absorption of the host cells of Chlamydia. When these granules are mixed with granules of drug prepared without the excipient, the uncombined granules provide the immediate effect, and the drug excipient granules provide extended action. The granule mix may be formulated as tablets or capsules. Hydrophilic cellulose polymers such as HPMC (hydroxylpropylmethylcellulose) are commonly used as the excipient base in tablet matrix systems.

A third suitable extended release system for a Chlamydia activating agent is directed to embedding drug in an inert plastic matrix. The Chlamydia activating agent is granulated with an inert plastic material such as polyethylene, polyvinyl acetate, or polymethacrylate, and the granulation is compressed into tablets. This creates a matrix that retains it shape during leaching of the drug and during its passage through the GI-tract. The drug is slowly released from the inert plastic matrix by diffusion. An immediate-release portion of the drug may be compressed onto surface of the tablet.

A fourth suitable extended release system for a Chlamydia activating agent is directed to an osmotic pump. A quite sophisticated extended release formulation for the Chlamydia activating agent is the oral osmotic pump drug delivery system (Oros system developed by Alza). This system is composed of a core tablet surrounded by a semipermeable membrane coating having a 0.4 mm diameter hole produced by laser beam. The core tablet has two layers, one containing the drug (the active layer) and the other containing a polymeric osmotic agent (the push layer). When the tablet is swallowed, the semipermeable membrane permits water to enter from the patient's stomach into the core tablet, dissolving or suspending the drug. As pressure increases in the osmotic layer, it pumps the drug solution out of the laser beam drilled hole. The drug release rate can be altered by changing the surface area, thickness or composition of the membrane, and/or diameter of the hole. The drug release rate is not affected by gastrointestinal acidity, alkalinity, fed conditions or gastrointestinal motility.

An extended release oral formulation for the Chlamydia activating agent provides a number of clear benefits compared to conventional tablets and capsules. An increased effect of the Chlamydia activating agent due to access to all cells in the GI tract hosting Chlamydia type microorganisms is obtained. The dosing frequency is reduced, while the patient obtains enhanced convenience and compliance and a reduction of overall health costs is obtainable. Also, reduction in adverse side effects will be reached. For example, high doses of tryptophan have been shown, on occasion, to produce intestinal discomfort and nausea. It is also known that high doses of tryptophan which exceed the intestinal absorption efficiency can increase the risk of toxic reactions in the intestinal tract due to bacterial action, which produces substances such as indoxylsulfate.

A second aspect of the invention provides a method for diagnosing IBS comprising determining the presence of Chlamydia bacteria in the enteroendocrine cells of the bowel, wherein the presence of Chlamydia bacteria in the enteroendocrine cells of the bowel is indicative of IBS. Conveniently, diagnosis can be carried out by using a detectable reagent that is specific for Chlamydia for detecting Chlamydia in a sample comprising enteroendocrince cells from the patient. One embodiment of the method for diagnosis comprises the use of mouse monoclonal antibody with clone number ACI directed against Chlamydia lipopolysaccaride. One embodiment of the method for diagnosis comprises the use of mouse monoclonal antibody with clone number BIOD 166 directed against Chlamydia trachomatis major outer membrane protein (MOMP). One advantage with this method is that it provides for a non-subjective and straightforward diagnosis. In one embodiment, this method for diagnosis is combined with Rome criteria.

A third aspect of the invention provides use of the mouse monoclonal antibody with clone number ACI directed against Chlamydia lipopolysaccaride for the diagnosis of IBS.

A fourth aspect of the invention provides the use of mouse monoclonal antibody with clone number BIOD 166 directed against Chlamydia trachomatis major outer membrane protein (MOMP) for the diagnosis of IBS.

### EXEMPLIFYING PART OF THE DESCRIPTION

### Example 1

This is a study of archived biopsy material from patients with IBS and control patients.

### Subjects and Methods

### Patients

A total of 61 females and 4 males with a median age of 48 (range 22-67) years, who fulfilled the Rome-II criteria for IBS⁴ were investigated. All of them had severe symptoms of IBS and 26 patients also exhibited abnormalities on small bowel manometry, thus qualifying for a pathophysiological diagnosis of enteric dysmotility.¹⁸

### Controls

Our control group comprised 42 persons (29 females) in whom IBS and all other functional bowel disorders had been excluded by medical interview and by use of a standardized questionnaire for the Rome-II symptom criteria. Ten controls (7 females) were obese but otherwise healthy. The median age of the controls was 36 (range 19-60) years.

### Questionnaire

42 patients and 32 healthy volunteers filled out a questionnaire about their past history of urogenital infections (recurrent urinary tract infections, chronic urethritis, salpingitis, ectopic pregnancy), bacterial enteritis and long-lasting upper and/or lower respiratory tract infections. None of them had any current symptoms indicating genital or respiratory Chlamydia infections.

### Full Thickness Jejunum Biopsy

Previously obtained full-thickness biopsy specimens were available in 62/65 patients. The biopsies had been taken from the proximal jejunum using a laparoscopy-assisted procedure described by Tornblom et al.⁹ Ten obese controls underwent full thickness biopsy of the jejunum at the time of gastric by-pass surgery.

### Mucosa Biopsy

Mucosa specimens from the proximal jejunum were taken with a Watson capsule in 32 controls and 6 patients. The Watson capsule was swallowed by the patient and brought by peristalsis to a position distal to the ligament of Treitz as determined by fluoroscopy. We also analyzed endoscopic biopsies from the duodenum in 18 patients where such material was available.

### Immunofluorescence

Expression of Chlamydia antigens was investigated using immunofluorescence on paraffin sections fixed in 10% formalin. Sections 5 μm thick were cut on a microtome and mounted on poly-L-lysine-treated microscope slides. Immunofluorescence was performed using a genus-specific FITC conjugated mouse monoclonal antibody to Chlamydia lipopolysaccharide (LPS) with Evans blue as a counter stain (RDI-PROAC1FT, Fitzgerald Industries International, Concord, Mass., USA). We also used mouse monoclonal antibody to Chlamydia trachomatis major outer membrane protein (MOMP) as primary antibody (GeneTex, Inc, USA) and polyclonal rabbit anti-mouse antibody-FITC conjugated (Dako, Glostrup, Danmark) as a secondary antibody. Cultured C. trachomatis served as positive controls. Different cell types were characterized using as primary antibodies rabbit polyclonal antibodies to Chromogranin A (Abcam, Cambridge, UK) for enteroendocrine cells, rabbit polyclonal antibodies to CD117 (Dako, Glostrup, Danmark) for mast cells, rabbit polyclonal antibodies to CD68 (Santa Cruz Biotechnology Inc, Santa Cruz, Calif.) for macrophages and rabbit monoclonal antibodies to CD11c (Abcam, Cambridge, UK) for dendritic cells and goat anti-rabbit antibodies conjugated with Alexa Fluor 568 (Invitrogen Corp., Carlsbad, Calif.) as secondary antibody.

Immediately before staining, the slides were warmed to 37° C. in an incubator for 30 minutes, and then deparaffinized in xylene and rehydrated in a grading series of alcohol dilutions and Tris-buffered saline (TBS) plus 0.1% Tween-20. After pre-treatment of the sections, 0.1% bovine serum diluted in TBS was applied for 20 minutes in a humidified chamber at room temperature in order to block non-specific binding.

Stained sections were examined using a Universal Laser Scanning Confocal Microscope System Leica TCS and a Fluorescent Microscope System Leica DMRXA (Leica Microsystems GmbH, Wetzlar, Germany). Two independent investigators, who were unaware of clinical data, made the final assessment of slides.

### Western Blotting

Frozen biopsies from 4 patients previously positive for Chlamydia LPS staining, were examined by Western blotting. HeLa cells infected with C. trachomatis served as positive control and we used non-infected HeLa cells as negative control. Equivalent amounts of protein from each specimen were loaded onto a sodium dodecyl sulphate-polyacrylamide gel. After electrophoresis, samples were transferred to nitrocellulose membranes. We used a mouse monoclonal antibody to C. trachomatis LPS (AbD Serotec, Oxford, UK) as primary antibody and goat anti-mouse antibody conjugated to horseradish peroxidase (BioRad, Herculaes, Calif., USA) as secondary antibody.

### Real-Time PCR

We used a real-time PCR assay developed by Everett et al. (1) which targets the 23S ribosomal DNA, and detects all members of the family Chlamydiaceae. An internal amplification control was included to monitor possible inhibition of the PCR. DNA from frozen biopsies, taken from 4 patients previously positive for Chlamydia LPS staining, was extracted using a Qiagen minikit according to the tissue protocol (QIAGEN Inc, Valencia, US). Extracted DNA was quantified and quality controlled using a Nanodrop spectrophotometer before subjected to PCR (Nanodrop Technologies, Wilmington, Del., USA).

### Transmission Electron Microscopy (TEM)

New biopsies from the distal duodenum of 4 patients previously positive for Chlamydia LPS staining were fixed in 2% glutaraldehyde+0.5% paraformaldehyde in 0.1M sodiumcacodylate buffer containing 0.1M sucrose and 3 mM CaCl₂, pH 7.4 at room temperature for 30 min followed by 24 hours at 4° C. Specimens were rinsed in 0.15 M sodiumcacodylate buffer containing 3 mM CaCl₂, pH 7.4 postfixed in 2% osmium tetroxide in 0.07 M sodiumcacodylate buffer containing 1.5 mM CaCl₂, pH 7.4 at 4° C. for 2 hour, dehydrated in ethanol followed by acetone and embedded in LX-112 (Ladd, Burlington, Vt., USA). Semi-thin sections were cut and stained with toluidin blue and used for light microscopic analysis. Ultra-thin sections were contrasted with uranyl acetate followed by lead citrate and examined in a Tecnai 10 transmission electron microscope at 80 kV. Digital images were taken by using a MegaView III digital camera (Soft Imaging System, GmbH, Munster, Germany) (Park, C. B., Asin-Cayuela, J., Camara, Y., Shi, Y., Pellegrini, M., Gaspari, M., Wibom, R., Hultenby, K., Erdjument-Bromage, H., Tempst, P., Falkenberg, M., Gustafsson, C. M., Larsson, N-G. MTERF3 is a negative regulator of mammalian mtDNA transcription Cell 2007, 130(2): 273-85)

### Serological Methods

We used ELISA plus medac kits (Medac, Hamburg, Germany) to detect IgA and IgG antibodies in serum towards C. trachomatis. The cHSP60-IgG-ELISA kit from the same manufacturer was used for detection of antibodies against a Chlamydia-specific 60-kDa heat shock protein (cHSP60) in serum. All tests were done according to the manufacturer's instructions.

### Statistical Analysis

We used Fisher's exact test for comparisons of proportions. The size of the study group was determined from an assumption that LPS positivity would be no greater than 20% among controls and no less than 70% among patients. In order to detect such a difference between patients and controls at p<0.01 with power >90% we needed to include at least 24 patients and 24 controls.

### Ethical Considerations

All parts of the study were approved by the Regional Ethical Review Board in Stockholm.

### Results

We analysed full thickness jejunum biopsies from 62/65 patients with severe IBS. Immunofluorescence (IFL) staining showed that 56/62 patients were positive for Chlamydia LPS in full thickness jejunum biopsies. Positive stains occurred mostly in intraepithelial cells. No Chlamydia LPS-positive cells were found in deeper layers of the bowel wall such as muscularis propria or the enteric nervous system. We therefore investigated if findings could be confirmed in biopsies from small bowel mucosa and analyzed biopsies obtained by Watson capsule and duodenal biopsies taken during gastroscopy in 22 of our patients with IBS. Finally 58/65 (89%) patients were positive in at least one biopsy. Six women and one man with IBS were negative in all biopsies. We confirmed the presence of Chlamydia LPS in patients with Western blotting analysis.

Seventy-nine percent of Chlamydia LPS positive biopsies were also positive to Chlamydia trachomatis MOMP. In 90% of positive biopsies from patients Chlamydia LPS was localised to mucosal cells at the level of crypts. Double staining with antibodies to chromogranin A showed that infected cells were enteroendocrine cells. In 69% of biopsies Chlamydia LPS was present in subepithelial cells. Staining with antibodies to CD117, CD11c and CD68 revealed that Chlamydia LPS was present mostly in macrophages and some mast cells.

Only 6/42 (14%) controls (5 women) were positive for Chlamydia LPS, mostly in subepithelial cells. Thirty three percent of LPS positive controls (2/6) were positive to Chlamydia trachomatis MOMP. The difference between patients (58/65) and controls (6/42) was highly significant (p<0.001) and the odds ratio for mucosal Chlamydia LPS being indicative for presence of IBS was 49.7 (95% CI: 15.5-159.7). Biopsies were analysed by two independent investigators unaware of clinical data. The agreement between the two investigators regarding individual biopsies was 94%, whereas their agreement regarding patient classification was 100%.

We found small amounts of Chlamydia reticular bodies in the aberrant form in enteroendocrine cells in small bowel biopsies taken from 4 patients. On the other hand we were unable to confirm the presence of Chlamydia DNA in the same biopsies using both the 16S rRNA sequence and the 23 S ribosomal DNA as a target.

In order to determine if persistence of Chlamydia antigens could be predicted by serology, we analysed serum samples from 25 available patients and from 24 healthy volunteers for antibodies against C. trachomatis and cHSP60. Table 1 shows the presence of antibodies and self-reported infectious history among patients and controls. Patients more often than controls reported previous urogenital infections, bacterial enteritis, and long-lasting respiratory infections, but none of the IgA or IgG antibodies showed any difference between the two groups. Neither did we find significant differences in cHSP60 IgG antibodies between patients and controls (9/25 patients and 10/24 controls).

TABLE 1
Immunofluorescence (IFL) staining for Chlamydia, past history
of infections, and presence of antibodies in patients with severe
IBS and controls.

| | Patients | Controls | Significance |
|---|---|---|---|
| IFL staining | | | |
| Positive for Chlamydia LPS | 58/65 (89%) | 6/42 (14%) | p < 0.0001 |
| Past history of infections | | | |
| Uro-genital infections | 24/42 (57%) | 7/32 (22%) | p < 0.01 |
| Bacterial enteritis | 20/42 (48%) | 5/32 (16%) | p < 0.01 |
| Long-lasting upper | 24/42 (57%) | 6/32 (19%) | p < 0.001 |
| respiratory tract infections | | | |
| Antibodies | | | |
| C trachomatis IgA | 0/25 (0%) | 2/24 (8%) | NS |
| C trachomatis IgG | 7/25 (28%) | 8/24 (33%) | NS |
| cHSP60 IgG | 9/25 (36%) | 10/24 (42%) | NS |

Evidently C. trachomatis infection in the small bowel can be linked to the presence of IBS. LPS and MOMP antigens are found in small bowel mucosa and submucosa in the vast majority of patients with IBS, but rarely so in healthy controls. In the mucosa these antigens were only found in enteroendocrine cells (EEC) at the level of the crypts. Electron microscopy confirmed the presence of Chlamydiae in the cytoplasm of EEC, where reticulate bodies in the aberrant form that is characteristic for persistent infection were found. The odds ratio for the association between C. trachomatis antigens and IBS was exceptionally large and this suggests an important role for C. trachomatis in IBS.

The presence of C. trachomatis in small bowel mucosa has not been demonstrated in IBS patients before. However, it is known that C. trachomatis can persist in the intestinal mucosa of certain animals for a long time without infection-induced inflammation.¹⁴ Such persistence can be a consequence of tissue-specific evolutionary adaptations that developed at this site to dampen T-cell responsiveness against commensal bacteria and food antigens.

The finding of C. trachomatis in EEC points at previously unrecognized pathogenetic mechanisms in IBS. Enteroendocrine cells are known to play a pivotal role in the control of gut motility and secretion and increased numbers of EEC have been detected in patients that developed IBS after an acute gastroenteritis.¹⁹ It is yet unknown if infection alters the function of EEC in man but animal experiments using different models of enteric infection have shown pronounced changes in both numbers of EEC and their function.^{20,21} Serotonin-producing EEC may present an ideal location for Chlamydia due to the presence of tryptophan. Tryptophan is required for normal development in Chlamydia species and tryptophan metabolism has been implicated in Chlamydial persistence and tissue tropism.²² Host interferon gamma (IFNγ) promotes tryptophan degradation in infected cells and this may lead to reversible inhibition of Chlamydia developmental cycle and persistent infection.²³ Infection localised in enteroendocrine cells allows Chlamydiae to survive in the presence of IFNγ-induced tryptophan limitation. On the other hand those bacteria capable of synthesing tryptophan from other sources will be at an advantage. Genital serovars of C. trachomatis are capable of utilizing indole produced by enteric bacteria for the biosynthesis of tryptophan.

Although presence of bacteria could be confirmed by electron microscopy, we were unable to detect Chlamydial DNA in our patients using standard extraction and amplification protocols. It is possible that the standard methods we used for nucleic acid retrieval and amplification were inadequate for the detection of persistent infection. Another explanation for the contradictory results could be that Chlamydial antigens were remainders of a past, but no longer present infection.²⁵ The latter seems unlikely in light of the long-term presence of C. trachomatis antigens observed in several of our patients. Long-term presence of antigens is more likely to be attributable to replicating Chlamydiae residing in the diseased tissue.²⁶

We found Chlamydia antigens in macrophages and some mast cells. It is known that Chlamydia become spontaneously persistent following the infection of monocytes, which are the common host cells for organisms during persistent infection.²⁷⁻²⁹ Chlamydiae may participate in the maintenance of local immunological response and inflammation via infected monocytes/macrophages and also use them to spread from infected cells to other organs.³⁰

No conclusions about the responsible Chlamydia species could be drawn from serology in LPS-positive samples. Our findings regarding serology are in agreement with one previous study of IgG antibodies to C. trachomatis in IBS.¹⁵ The use of serology for the diagnosis of infections with C. trachomatis is hampered by a high prevalence of antibodies among controls, and the sensitivity may be limited due a sometimes dominating cellular immune response to infection with Chlamydia.³¹⁻³³

Some of our Chlamydia LPS positive patients did not show the presence of MOMP antigen. Chlamydiae downregulate the major outer membrane protein (MOMP) expression at the persistent state. Attenuated synthesis of MOMP, usually considered the immunodominant surface epitope of the organism, in combination with its almost exclusively intracellular location during persistence may help to provide some relative invisibility from immune surveillance.³¹ This means that antibodies against MOMP may no longer be present in cases of persistent infection.

We used controls in which the presence of any functional bowel disorder had been excluded using a Rome-II symptom questionnaire. The odds ratio for mucosal Chlamydia LPS being indicative for presence of IBS (49.0; 95% CI: 9.9-243) is much higher than any previously described pathogenetic marker in IBS.³⁴ Our study is the first to demonstrate presence of C. trachomatis antigens in human small intestine and to analyse this in small intestinal biopsies from healthy subjects. We confirmed the presence of Chlamydia reticulate bodies in enteroendocrine cells in patients with IBS. In summary, C. trachomatis antigens were detected in biopsies from patients with IBS in a significantly higher proportion than in controls. We confirmed the presence of Chlamydia in enteroendocrine cells in patients with IBS. Serum antibody levels could not predict presence of Chlamydia antigens in small bowel mucosa. Our results suggest that persistent C. trachomatis infection in enteroendocrine cells is an important factor for the development of IBS.

### Example 2

A woman b. 1965 presented in 2005 with abdominal pain and vomiting after traveling abroad. Investigation of infection proved negative and the condition was characterized as a post-infectious functional disorder. The patient became ill again in 2006 with worsened pain symptoms and disturbed bowel function. Severe pains and abdominal distension in connection with food intake but also between meals were observed. Following hospitalization and treatment with analgesics, the patient underwent gastroscopy where a biopsy was taken. Histology indicated the presence of Chlamydia antigen in enterendocrine cells and in macrophages. The patient became partly symptom free following treatment with prednisolon and erythromycin. Due to the response to macrolide antibiotics, a persistent Chlamydia trachomatis infection in the bowel was suspected. Therefore, in December 2008, an attempt to eradicate the infection was made with a combination of azithromycin (500 mg twice per week) and rifabutin (150 mg). During the first ten days, tryptophan (250 mg four times) was administered, and prednisolon was administered in a decreasing dose starting at 30 mg per day. There was no improvement during the first two weeks of the treatment, but a slight improvement was subsequently noticed. The treatment was stopped after 8 weeks. At this time, the patient still had pains but increased 6 kg in weight, indicating improved bowel function. Gastroscopy was carried out again in March 2009. The patient is currently (April 2009) being weaned of analgesics. Biopsies from the duodenum were negative with regard to Chlamydia antigen at this time. This case is likely the first case where an etiologic agent for a severe bowel disease has been identified and removed by the combination of antibiotics and adjuvant treatment with steroids and tryptophan.

### REFERENCES

1. Drossman D A, Li Z, Andruzzi E, et al. U.S. householder survey of functional gastrointestinal disorders. Prevalence, sociodemography, and health impact. Dig Dis Sci 1993; 38(9):1569-80.
2. Agreus L, Svardsudd K, Nyren O, Tibblin G. The epidemiology of abdominal symptoms: prevalence and demographic characteristics in a Swedish adult population. A report from the Abdominal Symptom Study. Scand J Gastroenterol 1994; 29(2):102-9.
3. Mearin F, Badia X, Balboa A, et al. Irritable bowel syndrome prevalence varies enormously depending on the employed diagnostic criteria: comparison of Rome II versus previous criteria in a general population. Scand J Gastroenterol 2001; 36(11):1155-61.
4. Thompson W G, Longstreth G F, Drossman D A, Heaton K W, Irvine E J, Muller-Lissner S A. Functional bowel disorders and functional abdominal pain. Gut 1999; 45 Suppl 2:1143-7.
5. White B V, Jones C M. Mucous colitis: a delineation of the syndrome with certain observations on its mechanism and on the role of emotional tension as a precipitating factor. Ann Intern Med 1940; 14:854-72.
6. Chaudhary N A, Truelove S C. The irritable colon syndrome. A study of the clinical features, predisposing causes, and prognosis in 130 cases. Q J Med 1962; 31:307-22.
7. Whitehead W E, Bosmajian L, Zonderman A B, Costa P T, Jr., Schuster M M. Symptoms of psychologic distress associated with irritable bowel syndrome. Comparison of community and medical clinic samples. Gastroenterology 1988; 95(3):709-14.
8. Smith R C, Greenbaum D S, Vancouver J B, et al. Psychosocial factors are associated with health care seeking rather than diagnosis in irritable bowel syndrome. Gastroenterology 1990; 98(2):293-301.
9. Tornblom H, Lindberg G, Nyberg B, Veress B. Full-thickness biopsy of the jejunum reveals inflammation and enteric neuropathy in irritable bowel syndrome. Gastroenterology 2002; 123(6):1972-9.
10. Tornblom H, Holmvall P, Svenungsson B, Lindberg G. Gastrointestinal symptoms after infectious diarrhoea, a 5-year follow-up in a Swedish cohort of adults. Clin Gastroenterol Hepatol 2007; (in press).
11. Prior A, Wilson K, Whorwell P J, Faragher E B. Irritable bowel syndrome in the gynecological clinic. Survey of 798 new referrals. Dig Dis Sci 1989; 34(12):1820-4.
12. Zondervan K T, Yudkin P L, Vessey M P, et al. Chronic pelvic pain in the community—symptoms, investigations, and diagnoses. Am J Obstet Gynecol 2001; 184(6):1149-55.
13. Williams R E, Hartmann K E, Sandler R S, Miller W C, Savitz L A, Steege J F. Recognition and treatment of irritable bowel syndrome among women with chronic pelvic pain. Am J Obstet Gynecol 2005; 192(3):761-7.
14. Doughri A M, Storz J, Altera K P. Mode of entry and release of Chlamydiae in infections of intestinal epithelial cells. J Infect Dis 1972; 126(6):652-7.
15. Francis C, Prior A, Whorwell P J, Morris J. Chlamydia trachomatis infection: is it relevant in irritable bowel syndrome? Digestion 1998; 59(2):157-9.
16. Wilkinson N Z, Kingsley G H, Sieper J, Braun J, Ward M E. Lack of correlation between the detection of Chlamydia trachomatis DNA in synovial fluid from patients with a range of rheumatic diseases and the presence of an antiChlamydial immune response. Arthritis Rheum 1998; 41(5):845-54.
17. Witkin S S. Immunological aspects of genital Chlamydia infections. Best Pract Res Clin Obstet Gynaecol 2002; 16(6):865-74.
18. Wingate D, Hongo M, Kellow J, Lindberg G, Smout A. Disorders of gastrointestinal motility: towards a new classification. J Gastroenterol Hepatol 2002; 17 Suppl:S1-14.
19. Dunlop S P, Jenkins D, Neal K R, Spiller R C. Relative importance of enterochromaffin cell hyperplasia, anxiety, and depression in postinfectious IBS. Gastroenterology 2003; 125(6):1651-9.
20. Wheatcroft J, Wakelin D, Smith A, Mahoney C R, Mawe G, Spiller R. Enterochromaffin cell hyperplasia and decreased serotonin transporter in a mouse model of postinfectious bowel dysfunction. Neurogastroenterol Motil 2005; 17(6):863-70.
21. O'Hara J R, Skinn A C, MacNaughton W K, Sherman P M, Sharkey K A. Consequences of Citrobacter rodentium infection on enteroendocrine cells and the enteric nervous system in the mouse colon. Cell Microbiol 2006; 8(4):646-60.
22. Akers J C, Tan M. Molecular mechanism of tryptophan-dependent transcriptional regulation in Chlamydia trachomatis. J Bacteriol 2006; 188(12):4236-43.
23. Fehlner-Gardiner C, Roshick C, Carlson J H, et al. Molecular basis defining human Chlamydia trachomatis tissue tropism. A possible role for tryptophan synthase. J Biol Chem 2002; 277(30):26893-903.
24. Batteiger B E, Fraiz J, Newhall W J, Katz B P, Jones R B. Association of recurrent Chlamydial infection with gonorrhea. J Infect Dis 1989; 159(4):661-9.
25. Wyrick P B, Knight S T, Paul T R, Rank R G, Barbier C S. Persistent Chlamydial envelope antigens in antibiotic-exposed infected cells trigger neutrophil chemotaxis. J Infect Dis 1999; 179(4):954-66.
26. Beatty W L, Morrison R P, Byrne G I. Persistent Chlamydiae: from cell culture to a paradigm for Chlamydial pathogenesis. Microbiol Rev 1994; 58(4):686-99.
27. Hogan R J, Mathews S A, Mukhopadhyay S, Summersgill J T, Timms P. Chlamydial persistence: beyond the biphasic paradigm. Infect Immun 2004; 72(4):1843-55.
28. Peters J, Hess S, Endlich K, et al. Silencing or permanent activation: host-cell responses in models of persistent Chlamydia pneumoniae infection. Cell Microbiol 2005; 7(8):1099-108.
29. Rihl M, Kohler L, Klos A, Zeidler H. Persistent infection of Chlamydia in reactive arthritis. Ann Rheum Dis 2006; 65(3):281-4.
30. Airenne S, Surcel H M, Alakarppa H, et al. Chlamydia pneumoniae infection in human monocytes. Infect Immun 1999; 67(3):1445-9.
31. Inman R D, Whittum-Hudson J A, Schumacher H R, Hudson A P. Chlamydia and associated arthritis. Curr Opin Rheumatol 2000; 12(4):254-62.
32. Bas S, Muzzin P, Ninet B, Bornand J E, Scieux C, Vischer T L. Chlamydial serology: comparative diagnostic value of immunoblotting, microimmunofluorescence test, and immunoassays using different recombinant proteins as antigens. J Clin Microbiol 2001; 39(4):1368-77.
33. Sieper J, Braun J. Diagnosis and antibiotic treatment of reactive arthritis. Dtsch Med Wochenschr 2002; 127(37):1893-6.
34. Ohman L, Simren M. New insights into the pathogenesis and pathophysiology of irritable bowel syndrome. Dig Liver Dis 2007; 39(3):201-15.

1.-13. (canceled)

14. A method of treating the irritable bowel syndrome (IBS) comprising, administering during a pre-treatment period, an effective amount of a Chlamydia activating agent, and, during a following treatment period, administering one or several antibiotics selected from macroildes and benzoxazinorifamycins in a dose with efficacy for treating a virulent Chlamydia infection.

15. A method according to claim 14, wherein in the Chlamydia activating agent is tryptophan or a steroid.

16. A method according to claim 15, wherein the steroid is at least one selected from the group consisting of cortisone, prednisolone and budesonide.

17. A method according to claim 14, wherein the antibiotic is at least one selected from the group consisting of rifalazil, azithromycin and erythromycin.

18. A method according to claim 14, wherein the pre-treatment period extends for at least three days.

19. A method according to claim 14, wherein the activating agent is tryptophan and the antibiotic is rifalazil.

20. A method according to claim 14, wherein the Chlamydia activating agent is administered in an extended release formulation.

21. A method according to claim 20, wherein the extended release formulation comprises barrier coated beads, granules or microspheres comprising the Chlamydia activating agent.

22. A method according to claim 20, wherein the extended release formulation comprises the Chlamydia activating agent embedded in a slowly eroding or hydrophilic matrix system.

23. A method according to claim 20 wherein the extended release formulation comprises the Chlamydia activating agent embedded in inert plastic matrix.

24. A method according to claim 20, wherein the extended release formulation comprises the Chlamydia activating agent in a layer coated by semipermeable membrane with a hole in order to create an oral osmotic pump system.

25. A method for diagnosis of irritable bowel syndrome (IBS) comprising determining the presence of Chlamydia bacteria or Chlamydia antigen in the enteroendocrine cells of the bowel, wherein the presence of Chlamydia bacteria or Chlamydia antigen in the enteroendocrine cells of the bowel is indicative of IBS.

26. The method for diagnosis according to claim 25 comprising the use of the mouse monoclonal antibody with clone number ACI directed against Chlamydia lipopolysaccaride.

27. The method for diagnosis according to claim 25 comprising the use of the mouse monoclonal antibody with clone number BIOD 166 directed against Chlamydia trachomatis major outer membrane protein (MOMP).

28.-29. (canceled)
